(19) United States
(12) Patent Application Publication
(10) Pub. No.: US 2011/0065919 A1
(43) Pub. Date: Mar. 17, 2011

(54) 5-Substituted Indol-3-Carboxylic Acid Derivatives Exhibiting Antiviral Activity a Method for the Production and Use Thereof

(75) Inventors: Jury G. VERKHOVSKY, Obninsk (RU); Nina G. TSYSHKOVA, Obninsk (RU); Rakhimdzhan A. ROZIEV, Obninsk (RU); Anatoly F. TSYB, Obninsk (RU); Anna Ya. GONCHAROVA, Obninsk (RU); Fedor A. TROFIMOV, Obninsk (RU)

(73) Assignee: OOO "BINATEKH", Obninsk (RU)

(21) Appl. No.: 12/771,247

(22) Filed: Apr. 30, 2010

Related U.S. Application Data
(63) Continuation of application No. PCT/RU2008/000629, filed on Oct. 1, 2008.

(30) Foreign Application Priority Data
Oct. 31, 2007 (RU) 2007140220

(51) Int. Cl. C07D 413/14; C07D 209/42; C07D 401/06; C07D 403/12; C07D 413/06

(52) U.S. Cl. 544/82; 548/492; 546/201; 544/373; 544/144

(57) ABSTRACT

The invention relates to novel antiviral compounds of general formula (I), where B is —N(R)2 or —O—(CH2)nN(R)2 groups, in which n is a whole number selected from 0, 1, 2, 3 and 4, each R is independently selected from C1-4 alkyl and can be identical or different, or both groups R together with a nitrogen atom, to which they are bonded, form a 5-6-membered heterocyclic ring containing 1-2 heteroatoms selected from nitrogen, oxygen and sulphur, such as pyrrolidine, piperidine, piperazine, morpholine or thyomorpholine, at which each of above-mentioned heterocyclic rings can be substituted by C1-4 alkyl, phenyl, benzyl, phenetyl, a carbonylamino —COOC1-4 alkyl group or the carbonylamino —COOC1-4 alkyl group and phenyl, which also can be substituted and have substituents selected from halogen, C1-4 alkyl, C1-4 alkoxy, and alkyl in said groups can be linear or branched; R1 is C1-4 alkoxy, phenyl optionally substituted by C1-4 alkyl or C1-4 alkoxy, halogen atoms, naphthyl; R2 is C1-4 alkyl, —S-phenyl, —S-benzyl, —O-phenyl, O-benzyl, wherein in each of the above-mentioned groups the phenyl ring is optionally substituted by C1-4 alkyl, C1-4 alkoxy, halogen atoms, or R2 is an —NR3R4 group, in which R3 and R4, each is independently selected from C1-4 alkyl and can be identical or different, or both R3 and R4 groups together with a nitrogen atom, to which they are bonded, form a 5-6-membered nitrogen-containing heterocyclic ring having the above mentioned value for the N(R)2 group; X is hydrogen or a halogen atom selected from Br, Cl, and I or pharmaceutically acceptable salts thereof. Intermediate products of general formula (II) and a method for producing the inventive compounds are also disclosed

### RELATED APPLICATIONS

This application is a Continuation of International Application No. PCT/RU2008/000629, filed Oct. 1, 2008, which claims priority to Russian Patent Application No. RU 2007140220, filed Oct. 31, 2007, both of which are incorporated herein by reference in their entirety.

### FIELD OF THE INVENTION

The invention relates to novel 5-substituted hydroxyindole-3-carboxylic acid derivatives with antiviral activity that may find application for the prophylaxis and treatment of a prevalent viral disease such as influenza.

### BACKGROUND OF THE INVENTION

As a rule, existing antiviral drugs are active only in relation to specific viruses. More than a thousand variants have already been discovered at the present time; further, about a half of them represent a danger to humans. Therefore, the creation of new antiviral drugs is simply a necessity. The mechanism of action of contemporary antiviral drugs consists in the blocking of one of the stages of reproduction of viruses in the cells of the virus carrier (man) that include the stage of attachment of the virus to the cell and penetration into it, the insertion of the virus's nucleic acid into the genome of the host cell, the synthesis of its own DNA and RNA, and the synthesis and assembly of the intrinsic proteins of the virus. The majority of available antiviral drugs are analogs of nucleosides. They are effective, but recurrences have been found in a number of patients after their use, and the rebound phenomenon, leading to exacerbation of the illness, is possible. In addition, the development of resistance of the viruses to them is characteristic of a number of antiviral drugs. All of the factors enumerated are evidence of the fact that only the creation of new antiviral drugs can ensure progress in the treatment of viral diseases.

There are a large number of publications describing 6-halogen-5-hydroxyindol-3-carboxylates.

The best known and most effective compound exhibiting antiviral action is Arbidol (1-methyl-2-phenylthiomethyl-3-carbethoxy-4-dimethylaminomethyl-5-hydroxy-6-bromindol) (see, for example, RU patent 2033156). Arbidol is capable of stimulating various immunity functions, is well tolerated when used for the prophylaxis and treatment of viral infections. However, arbidol is insufficiently efficacious against some strains of viruses, for example, strains of the viruses of influenza A and B.

N. G. Tsyshkova et al., in the article, “Synthesis of 1,2,3,4-tetrahydropyrazino[1,2-a]indol derivatives and their pharmacological investigation,” Pharmaceutical Chemistry Journal, 1992, 26 (9-10), describe compounds of the general formula (3)

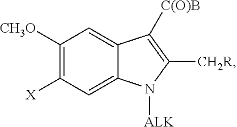

where X is H, Br, B is OEt, R is H, and Alk is CH₂CH₂Br, as intermediates for the production of biologically active compounds.

G. S. Gadaginamath et al., in the Polish Journal of Chemistry, 1997, 71 (7) 923-28, describe the synthesis and antibacterial activity of compounds of the general formula (3), where X is Br, R is OR, SR1, R2 (R is Ph, 2(4)Cl-Ph, β-naphthyl, etc., R1 is Ph, 4-ClPh, R2 is NMePh, 4-Br-PhNH, etc.), B is OEt, and Alk is n-butyl.

WO2006/42045A1 discloses the synthesis and properties of 6-F-indolcarboxylates as antagonists of chemokine CCR-5 receptors.

WO2007/014851A2 describes N-(C2-6-alkyl)indol-3-yl-carbonyl-piperazine and 3-carbonyl-piperidine derivatives, possibly substituted at indol position 2 by an alkylcarbonyl, aminoalkylcarbonyl, or alkyl group, possibly substituted by halogen atoms, amino, alkylcarbonyloxy, alkyl-, aryl-, or heteroarylcarbonylamino group. The known compounds can be used for the preparation of medicinal agents for the treatment of various diseases, for example, in the treatment of depression, hypertension, etc

UA 78317C2 describes derivatives of 1-[(indol-3-yl)carbonyl]piperazine unsubstituted at position 2 that may be used as analgesics, as well as pharmaceutical compositions and a method of producing the compounds.

JP 06-199784, published 19.07.1994a, describes arylethylamines of the following formula I:

Ar¹-CH₂CH₂—N—(R₂)—R₁, where Ar¹ is a group of formula II

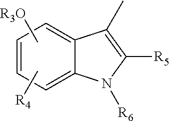

R₃ is H, OH, a halogen, a C₁₋₆ alkyl, or an aryl; R₄ is H, OH, a halogen, or a C₁₋₆ alkoxy; R₅ is H, a halogen, or a C₁₋₆ alkyl; R₆ is H or a C₁₋₆ alkyl; R₁ is a group of formula III —COR₇, where R₇ is for example, trifluoromethyl; R₂ is H or a C₁₋₆-alkyl. In particular, a compound such as N-[2-(5-methoxyindol-3-yl)ethyl-cyclopropyl carboxamide is disclosed, as well as their production and pharmaceutical compositions based on them. Said compounds are of low toxicity, exhibit excellent selective compatibility with the serotonin receptor, and can be used as a sedative and an anxiety-reducing agent as well as antipsychotic agents.

### SUMMARY OF THE INVENTION

The objective of the present invention is a search for novel 5-substituted indol-3-carboxylic acid derivatives exhibiting high antiviral activity and low toxicity. Further, the compounds of the invention exhibit virus-specific activity comparable with the activity of arbidol and surpass the activity of some known compounds with antiviral action.

The above and other features of the invention including various novel details of construction and combinations of parts, and other advantages, will now be more particularly described and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

According to the present invention, compounds of the general formula (I)

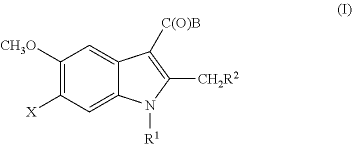

are proposed, where B represents an N(R)₂ group,

each R is selected independently from a C₁₋₄alkyl and may be the same or different,

or both R groups together with the nitrogen atom to which they are attached form a 5- to 6-member heterocyclic ring containing 1 to 2 heteroatoms, selected from nitrogen, oxygen, and sulfur, such as pyrrolidine, piperidine, piperazine, and morpholine. Further, each of said heterocyclic rings may be substituted by a C₁-alkyl, phenyl, benzyl, phenetyl, carbonylamino,

a COOC₁₋₄alkyl group or a COOC₁₋₄alkyl group and a phenyl. Further, the phenyl ring in each of the groups may have substituents selected from the following: a halogen, a C₁₋₄alkyl, or a C₁₋₄alkoxy, and the alkyl may be linear or branched; R¹ represents a C₁₋₄alkyl, a phenyl, possibly substituted by a C₁₋₄alkyl or a C₁₋₄alkoxy, by halogen atoms or a naphthyl;

R² represents a C₁₋₄alkyl, an S-phenyl, an S-benzyl, or an O-phenyl; therewith, in each of said groups the phenyl ring is possibly substituted by a C₁₋₄alkyl,

a C₁₋₄alkoxy, or halogen atoms, or

R² represents an N(R)₂ group in which each R is selected independently from a C₁₋₄-alkyl and may be the same or different,

or both R groups together with the nitrogen atom to which they are attached form a 5- to 6-membered nitrogen-containing heterocyclic ring having the above-mentioned value for the N(R)₂ group;

X represents hydrogen or a halogen atom selected from Br, Cl, or I, and their pharmaceutically acceptable salts.

The invention also relates to compounds of the general formula (VI)

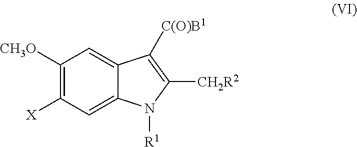

where the values X, R¹, and R² have the above-mentioned values and B¹ represents a hydroxy group, that are intermediate products for the production of compounds of formula (I).

Compounds of formula (I) and (VI) have not been previously produced and the antiviral activity of the compounds of formula (I) applied is unknown.

The invention also relates to methods of production of compounds of formula (I).

Compounds of formula (I) may be produced in accordance with the schemes indicated below:

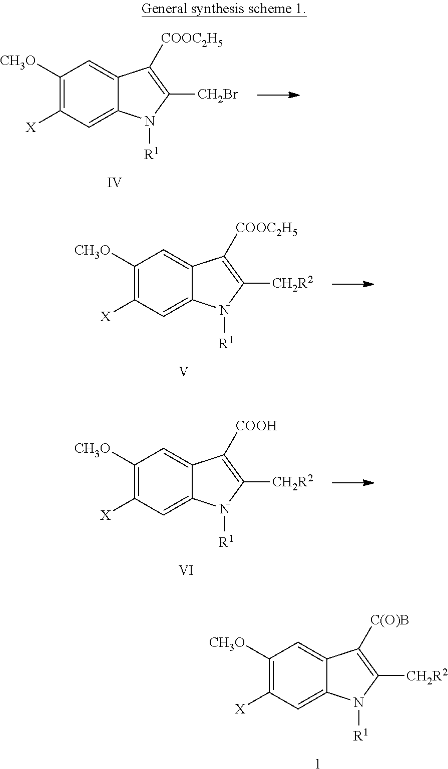

X, B, R¹, and R² have the values mentioned above for compounds (I).

According to general scheme 1, the lower alkyl ester of the corresponding N-substituted 2-bromomethyl-5-methoxy-6-haloindol-3-yl-carboxylic acid is subjected to interaction with an appropriate nucleophilic reagent selected from the appropriate thiophenol, phenol, or secondary amine; the ester group is then saponified to obtain the corresponding intermediately formed indol-3-yl-carboxylic acid. The latter is subjected to interaction with an appropriate secondary amine directly or after conversion into the corresponding halogenide. Addition of a dehydrating agent is possible when the directly intermediately formed acid is used in the reaction with the amine.

The formula I amide obtained thereby is isolated or, if necessary, converted to the salt. The salts may be prepared by the usual methods, for example, by treatment of the formula I compound with the corresponding acid.

Among the salts are the following, for example: hydrochlorides, mesylates, oxalates, sulfates, etc. In greater detail, the method is carried out as follows.

According to scheme 2 indicated below, compounds (1) and (2) may be obtained, where R² represents an S-phenyl, an S-benzyl, an O-phenyl, or —NR³R⁴; further, in each of said groups the phenyl ring may be substituted by a C₁₋₄alkyl,

a C₁₋₄alkoxy, or halogen atoms. X, N(R)₂, and R¹, R³, and R⁴ have the values mentioned above.

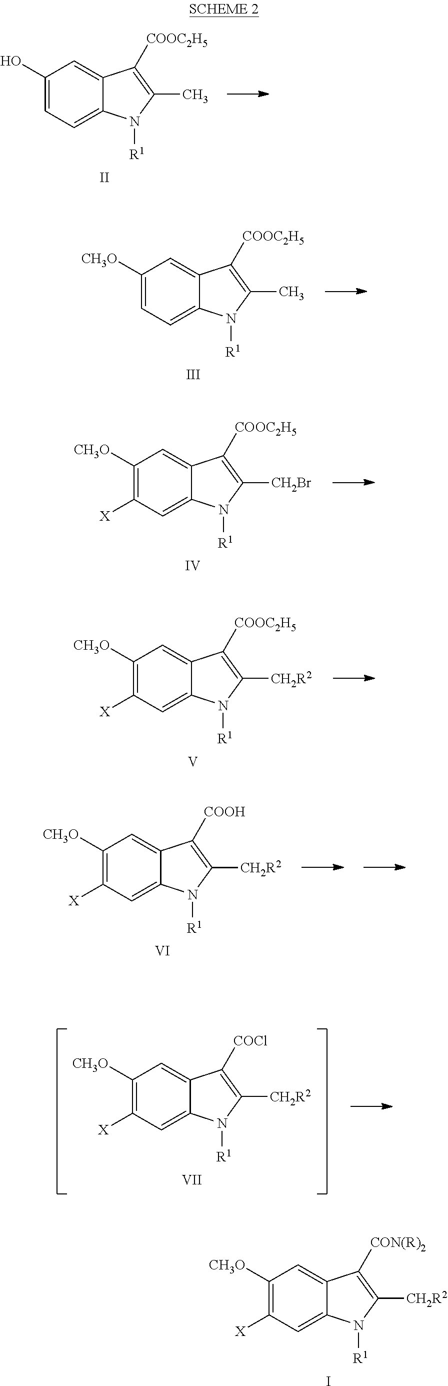

The method according to scheme 2 is run by starting from the ethyl ester of the corresponding N-substituted 2-methyl-5-hydroxyindol-3-yl-carboxylic acid (II) by methylation of the hydroxy group, with the following sequential bromination of the product obtained and interaction with a nucleophilic reagent selected from compounds permitting the production of the corresponding formula I compounds with the aforesaid value of R².

The ethyl ester of 6-bromo-2-bromomethyl-1-methyl-5-ethoxyindol-3-carboxylic acid, described in the literature (Chemistry of Heterocyclic Compounds, 1973, No. 3, pp. 308-311) which, following interaction with the appropriate nucleophilic reagents (amine, phenols, thiophenols), is subjected to saponification, may be used as the starting compound. The acids obtained, by interacting with thionyl chloride and then with secondary amines, yield the corresponding amides.

Synthesis according to scheme 2. The starting compound according to example 1- the ethyl ester of 6-bromo-1-methyl-5-methoxy-2-(phenylthio)indol-3-carboxylic acid (IV) is obtained by the method using the steps described in scheme 2 and disclosed by F. A. Trofimov et al. in Chemistry of Heterocyclic Compounds, 1973, No. 3, pp. 308-311.

### Example 1

### 6-Bromo-1-methyl-5-methoxy-2-(phenylthio)indol-3 carboxylic acid (VI)

A solution of 3.8 g (0.009 mole) of the ethyl ester of 6-bromo-1-methyl-5-methoxy-2-(phenylthio)indol-3-carboxylic acid, 5 g of caustic soda, and 3 mL of water in 100 mL of ethyl alcohol is boiled for 3 hours. It is partially evaporated in vacuo, water is added to dissolution of the salt, and acidified with concentrated hydrochloric acid while cooling. The precipitate is filtered and washed with water. Yield 3.4 g (92%). Melting point 213° C. (with decomposition from dioxane).

Found, % C, 52.97; H 3.94. C₁₈H₁₆BrNO₃S. Calculated, %: C, 53.91; H, 3.96.

Similarly obtained:

6-bromo-5-methoxy-1-phenyl-2-phenylthiomethyl-indol-3 carboxylic acid, melting point 200-202° C. (decomposition, from dioxane), found. %: C, 59.03; H, 4.22; C₂₃H₂₈BrNO₃S. Calculated, %: C, 58.96; H, 3.88.

### Example 2

### 6-Bromo-1-methyl-5-methoxy-2-(phenylthio)indol-3-carboxylic acid chloranhydride (VII)

To a suspension of 2.03 g (0.005 mole) of compound VI in 20 mL of dioxane at room temperature are added 2 mL of thionyl chloride and 1 drop of dimethylformamide. This is heated to dissolution in a water bath and left at room temperature for a day. The dioxane and the excess thionyl chloride are distilled off in vacuum; hexane is added to the residue. The precipitate of 6-bromo-1-methyl-5-methoxy-2-(phenylthio)indol-3-carboxylic acid chloranhydride (VII is filtered, washed in hexane, and used in the following step without purification.

### Example 3

### 1-{6-Bromo-1-methyl-5-methoxy-2-(phenylthio)methyl-1-H-indol-3-yl}-benzylpiperazine (A)

To a solution of 1.3 g (0.00306 mole) of VII in 10 mL of benzol are added 0.54 g (0.00306 mole) of 4-benzylpiperazine in 5 mL of benzol and 0.45 mL of triethylamine. This is left at room temperature for a day. The benzol is distilled off in vacuum; water is added to the oily residue, decanted twice with the oily precipitate. Ethanol is added to the residue, the mixture is cooled, and the precipitate filtered. Yield 1.35 g (78.4%). Melting point 167-169° C. (from acetone).

The hydrochloride is obtained by adding hydrochloric acid to a solution of the base in acetone; melting point 220° C. (from aqueous alcohol)

Found, %: C, 58.71; H, 5.24; N, 6.84; S 5.28. C₂₉H₃₁BrClN₃O₂S. Calculated, %: C, 57.96; H, 5.20; N 6.99; S 5.33.

Similarly obtained:
1-{6-Bromo-1-methyl-5-methoxy-2-(phenylthio)methyl-1-H-indol-3-yl]carbonyl}pyrrolidine (B), Melting point 150° C. (from alcohol).
1-{6-Bromo-1-methyl-5-methoxy-2-(phenylthio)methyl-1-H-indol-3-yl]carbonyl}piperidine (C), Melting point 165° C. (from alcohol and acetone).
1-{6-Bromo-1-methyl-5-methoxy-2-(phenylthio)methyl-1-H-indol-3-yl]carbonyl}morpholine, Melting point 147° C. (from alcohol).
1-{6-Bromo-1-methyl-5-methoxy-2-(phenylthio)methyl-1-H-indol-3-yl]carbonyl}methylpiperazine, Melting point 156° C. (from aqueous alcohol).
1-{6-Bromo-1-methyl-5-methoxy-2-(phenylthio)methyl-1-H-indol-3-yl]carbonyl}-4-phenylpiperazine, hydrochloride, Melting point 120° C. (from isopropyl alcohol).
1-{6-Iodo-1-methyl-5-methoxy-2-(phenylthio)methyl-1-H-indol-3-yl]carbonyl}-4-phenylpiperazine, Melting point 163° C. (from alcohol).
1-{6-Bromo-1-methyl-5-methoxy-1-phenyl-1-H-indol-3-yl]carbonyl}-4-phenyl-4-ethoxycarbonylpiperidine, Melting point 128° C. (from alcohol).
1-{[2-methyl-5-methoxy-1-phenyl-1-H-indol-3-yl]carbonyl}-4-phenyl-4-ethoxycarbonylpiperidine, Melting point 140° C. (from alcohol).
1-{[2-methyl-5-methoxy-1-phenyl-1-(4-methoxyphenyl)-1-H-indol-3-yl]carbonyl}-4-phenyl-4-carbethoxypiperidine, Melting point 130° C. (from alcohol).

Synthesis according to scheme 3

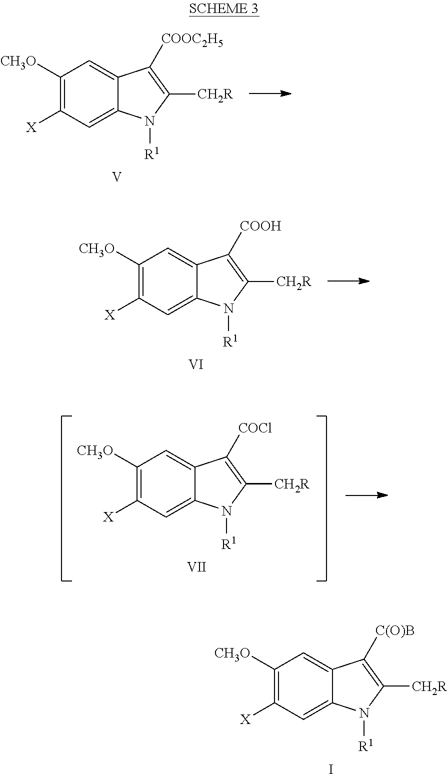

### Example 4

### 6-Bromo-1-methyl-5-methoxy-2-piperidinomethylindol-3-carboxylic acid hydrochloride (X)

A solution of 4.1 g (0.01 mole) of the ethyl ester of 6-Bromo-1-methyl-5-methoxy-2-piperidinomethylindol-3-carboxylic acid, 6.0 g of caustic soda, 3 mL of water and 60 mL of ethyl alcohol is boiled for 3 hours; then 10 mL of water is added and the mixture is acidified with concentrated hydrochloric acid to acidic reaction while cooling. The precipitate is filtered and recrystallized from aqueous alcohol. Yield 4.1 g (98%). Melting point 236-238° C. (from aqueous alcohol).

### Example 5

### 1-{6-Bromo-1-methyl-5-methoxy-2-piperidinomethyl-1-H-indol-3-yl]carbonyl}-piperidine (XII)

A mixture of 1.25 g (0.003 mole) of compound X, 2 mL of thionyl chloride, and 1 mL of dimethylformamide in 20 mL of dioxane is heated in a water bath for 3 hours. This is evaporated to dryness at reduced pressure, and the hydrochloride precipitate of the chloranhydride XI is washed with ether. To the precipitate obtained are added 15 mL of benzol and a mixture of 0.51 g (0.006 mole) of piperidine and 0.8 mL of triethylamine in 5 mL of benzol. The reaction mass is heated in a water bath for 2 hours, the triethylamine hydrochloride precipitate is filtered out and washed with hot benzol. The benzol solution is evaporated in vacuum and hexane is added. The product, settled as a precipitate, is filtered and washed with hexane. Melting point 156-157° C. (from a mixture of alcohol and hexane).

Yield 0.7 g (52%).
1-{6-Bromo-1-methyl-5-methoxy-2-morpholinomethyl-1-H-indol-3-yl]carbonyl}-morpholine (XII) is similarly obtained, Melting point 155° C. (from alcohol and hexane).
   Synthesis according to scheme 2 with the use of the corresponding hydroxy compounds as the nucleophilic reagent (for example, 4-chlorophenol, 4-hydroxypiperidine, etc.).

### Example 6

### The ethyl ester of 6-bromo-1-methyl-5-methoxy-2-(4′-chlorophenyloxymethyl)indolyl-3-carboxylic acid (XIII)

A mixture of 2.0 g (0.005 mole) of the ethyl ester of 6-bromo-1-methyl-5-methoxyindolyl-3-carboxylic acid, 0.13 g (0.01 mole) of 4-chlorophenol, and 3.5 g of anhydrous potassium carbonate in 30 mL of acetone are boiled for 7 hours. The mixture is cooled; the precipitate is filtered and carefully washed with water and alcohol. Yield 1.85 g (81.8%), melting point 159-160° C. (from acetone).

### Example 7

### 6-bromo-1-methyl-5-methoxy-2-(4′-chlorophenyloxymethyl)indolyl-3-carboxylic acid (XV)

A solution of 0.9 g (0.002 mole) of compound XIII, 1.2 g of caustic soda, 0.6 mL of water in 10 mL of ethyl alcohol, and 5 mL of acetone is boiled for 2 hours. Then 10 mL of water is added and while cooling this is acidified with concentrated hydrochloric acid. The precipitate is filtered and washed with water. Yield 0.78 g (92%). Melting point greater than 270° C. (decomposition).

Similarly obtained: 6-bromo-1-methyl-5-methoxy-2(4′-methoxyphenyloxymethyl)indolyl-3-carboxylic acid, Melting point 220° C. (from dioxane)

### Example 8

### 1-{6-Bromo-1-methyl-5-methoxy-2-(0.4′-chlorophenyloxy)-methyl-1-H-indol-3-yl]carbonyl}-4′-methylpiperazine

To a solution of 0.63 g (0.0015 mole) of compound XV in 10 mL of dioxane is added 1 mL of thionyl chloride. The dioxane and the excess thionyl chloride are distilled off from the chloranhydride obtained; heptane is added to the residue, the precipitate is filtered and dissolved in 10 mL benzol. To the solution is added 0.3 g (0.003 mole) of 4-methylpiperazine and the mixture is left for a day at room temperature. The mixture is evaporated and water is added to the residue. The precipitate is filtered and washed with water. Yield 0.5 g (65.7%).

Melting point 158-160° C. (from a mixture of ethylacetate and petroleum ether).

The following were similarly obtained:
1-{6-Bromo-1-methyl-5-methoxy-2-(4′-methoxyphenyloxymethyl)-1-H-indol-3-yl]carbonyl}-4′-benzylpiperazine, melting point 155° C. (from alcohol)
1-{6-Bromo-1-methyl-5-methoxy-2-(4′-methoxyphenyloxymethyl)-1-H-indol-3-yl]carbonyl}-4′-ethoxycarbonylpiperazine, melting point 163° C. (from iso-PrOH)
1-{6-Bromo-1-methyl-5-methoxy-2-(4′-methoxyphenyloxymethyl)-1-H-indol-3-yl]carbonyl}-4′-morpholine, melting point 149° C. (from alcohol)
1-{6-Bromo-1-methyl-5-methoxy-2-(4′-methoxyphenyloxymethyl)-1-H-indol-3-yl]carbonyl}-4′-(2-methylphenyl)piperazine, melting point 184° C. (from alcohol)
1-{6-Chloro-1-methyl-5-methoxy-2-(4′-methoxyphenyloxymethyl)-1-H-indol-3-yl]carbonyl}-4′-morpholine, melting point 165° C. (from alcohol)
1-{6-Bromo-1-methyl-5-methoxy-2-(4′-methoxyphenyloxymethyl)-1-H-indol-3-yl]carbonyl}-4′-phenylpiperazine, 175° C. (from alcohol)
1-{6-Bromo-1-methyl-5-methoxy-2-(4′-methoxyphenyloxymethyl)-1-H-indol-3-yl]carbonyl}-4′-methylpiperazine, melting point 143 (from alcohol)
1-{6-Bromo-1-methyl-5-methoxy-2-(4′-methoxyphenyloxymethyl)-1-H-indol-3-yl]carbonyl)-4′-(4-methylphenyl)piperazine, melting point 178° C. (from alcohol)
1-{6-Bromo-1-methyl-5-methoxy-2-(4′-methoxyphenyloxymethyl)-1-H-indol-3-yl]carbonyl}-4′-(4-aminocarbonyl)piperidine, melting point 163° C. (from hexane)
1-{6-Bromo-1-methyl-5-methoxy-2-(4′-methoxyphenyloxymethyl)-1-H-indol-3-yl]carbonyl}-4′-phenyl-4′-ethoxycarbonylpiperidine, melting point 92° C. (from hexane and iso-PrOH).

The structures of the compounds obtained were confirmed by the data of the PMR [proton magnetic resonance] spectra, elemental analysis, and the individuality by thin-layer chromatography on Silufol UV254 plates.

Tests of biological activity.

1. Effect of arbidol analogs on reproduction of the influenza virus in MDCK tissue culture cells

The cytotoxic action of the compounds were studied in preliminary experiments and maximal tolerated concentrations (MTC) were established (Table 1).
The tests of antiviral activity were carried out on the influenza A/New Calcdonia/20/99 (H1N1) virus reference strain under conditions of identical multiplicity of infection with the virus. Concentrations (μg/mL) inhibiting viral reproduction by 50% (MIC₅₀) were determined for the comparison drug (arbidol) and the compounds to be tested, and the chemotherapeutic index (ratio of the MTC to the MIC₅₀) was calculated; the activity of the substances at a concentration of 10 μg/mL was also studied (Table 1).

It can be seen from the data given in Table 1 that compounds A and B possess high specific activity, commensurable with the activity of arbidol, in relation to the influenza A virus reference strain (H1N1).

On the basis of the MIC₅₀ and the chemotherapeutic index, compounds A and B even surpassed arbidol somewhat. At a concentration of 10 μg/mL, however, compound A was inferior to arbidol in efficacy, while compound B did not differ from it. Compound C showed low activity.

TABLE 1
Inhibition by Arbidol Analogs of Reproduction of the Influenza
A/New Caledonia/20/99 (H1N1) Virus Reference Strain in MDCK
Tissue Culture Cells.

| | MTC of the | | | |
|---|---|---|---|---|
| | Compounds, | MIC₅₀, | Chemotherapeutic | Activity at |
| Compounds | μg/mL | μg/mL | Index | 10 μg/mL |
| Arbidol | 40 | 6.0 | 6.7 | 100.0 |
| C | 25 | 9.2 | 2.7 | 52.1 |
| B | 15 | 2.0 | 7.5 | 100.0 |
| A | 40 | 5.8 | 6.9 | 64.4 |

2. Determination of the efficacy of the compounds in the murine influenza pneumonia model.

The mice were infected intranasally under light ether anesthesia with the influenza A/Aichi/2/69 (H3N3) virus. The control animals did not receive any treatment, while the experimental animals were administered arbidol of compound A in a dose of 60 mg/kg/day 24 hours and 1 hour before inoculation, and then 24 hours after infection and on the following 4 days. The efficacy of the action of the compounds in the influenza pneumonia model were assessed according to the number of mice surviving after infection with the virus and the increase in mean life span

The mean life span of the mice was calculated according to the following formula: MSD [mean survival day]=Σ(f(d−1)n, where f is the number of mice dying on day d; the surviving mice were also included in f, and d in this case was 15; n is the number of mice in the group.

TABLE 2
Efficacy of the Compounds in the Murine Influenza Pneumonia
Model (A/Aichi/2/69 Virus).

| | | | | Mortality | Life Span |
|---|---|---|---|---|---|
| Compounds | n | Survived | Mortality, % | reduction, % | (Days) |
| Infected control | 16 | 3 | 81 | | 8.1 |
| Arbidol | 10 | 8 | 20 | 61 | 12.2 |
| A | 10 | 8 | 20 | 61 | 13.1 |

It can be seen from Table 2 that compound A is not inferior to arbidol, a known antiviral agent, by chemotherapeutic action testing for efficacy in the murine influenza pneumonia model.

While this invention has been particularly shown and described with references to preferred embodiments thereof, it will be understood by those skilled in the art that various changes in form and details may be made therein without departing from the scope of the invention encompassed by the appended claims.

1. Compounds of a general formula (I) 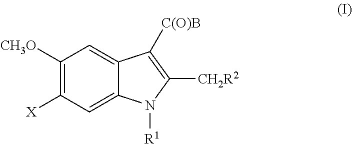
where B is an N(R)₂ group,
each R is selected independently from a C₁₋₄alkyl and each R is the same or different,
or both R groups, together with the nitrogen atom to which they are attached, form a 5- to 6-member heterocyclic ring containing 1 to 2 heteroatoms, selected from nitrogen, oxygen, and sulfur, such as pyrrolidine, piperidine, piperazine, or morpholine; further, each of said heterocyclic rings may be substituted by a C₁-alkyl, phenyl, benzyl, phenetyl, carbonylamino,
a COOC₁₋₄alkyl group or a COOC₁₋₄alkyl group and a phenyl, which also be substituted and have substituents selected from the following: a halogen, C₁₋₄alkyl,
or C₁₋₄alkoxy, and the alkyl in said groups may be linear or branched; R¹ represents a C₁₋₄alkyl, a phenyl, possibly substituted by a C₁₋₄alkyl, a C₁₋₄alkoxy, or by halogen atoms;
R² represents a C₁₋₄alkyl, an S-phenyl, an S-benzyl, or an O-phenyl; therewith, in each of said groups the phenyl ring is possibly substituted by a C₁₋₄alkyl,
a C₁₋₄alkoxy, or halogen atoms, or
R² represents an N(R)₂ group;
X represents hydrogen or a halogen atom selected from Br, Cl, or I, and their pharmaceutically acceptable salts.

2. Compounds of a general formula (II) 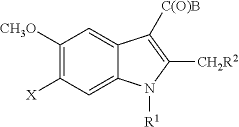
where B is a hydroxy group,
R¹ represents a C₁₋₄alkyl, a phenyl, possibly substituted by a C₁₋₄alkyl or a C₁₋₄alkoxy, or by halogen atoms;
R² represents a C₁₋₄alkyl, an S-phenyl, an S-benzyl, or an O-phenyl; therewith, in each of said groups the phenyl ring is possibly substituted by a C₁₋₄alkyl,
a C₁₋₄alkoxy, or halogen atoms, or
R² represents an N(R)₂ group in which each R is selected independently from a C₁₋₄alkyl and may be the same or different,
or both R groups together with the nitrogen atom to which they are attached form a 5- to 6-member nitrogen-containing heterocyclic ring having the above-mentioned value for the N(R)₂ group;
X represents hydrogen or a halogen atom selected from Br, Cl, or I.

3. A method for producing compounds of a general formula (I) 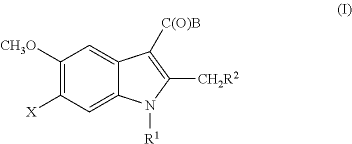
where the values of B, X, and R¹ are indicated in claim 1 and R² represents N(R)₂, distinguished by the fact that a compound of formula (IV) 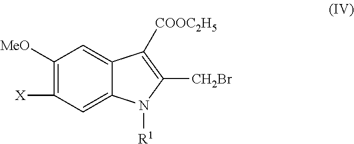
where the values of X and R are indicated above,
is subjected to interaction with an appropriate nucleophilic reagent selected from the appropriate phenol, thiophenol, or secondary amine, including the fragment N(R)₂, to obtain a compound of general formula (V) 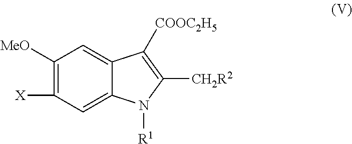
where the values of X, R′, and R² are indicated above,
in which the ester group is then saponified with production of an acid of general formula (VI) 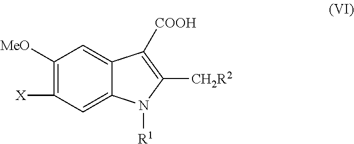
where the values of X, R¹, and R² are indicated above,
and the acid of formula (VI) obtained, directly or after conversion into the corresponding haloid anhydride, is subjected to interaction with an appropriate secondary amine, including the fragment N(R)₂, with the generation of a compound of formula (I) in the free form or in the form of a pharmaceutically acceptable salt.

4. A method according to claim 3, distinguished by the fact that a compound is used of general formula (IV), obtained by methylation of a compound of general formula (II), 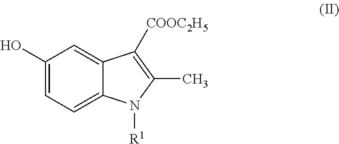
in which X represents hydrogen and R¹ has the values indicated above, with the production of the corresponding ester of 5-methoxyindol-3-yl-carboxylic acid of formula (III) 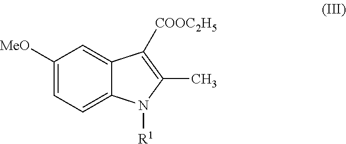
where R¹ has the values indicated above,
and its subsequent bromination.

5. A method according to claim either of claim 3, distinguished by the fact that an acid of formula VI 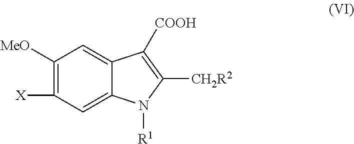
where R¹ and R² have the values indicated above,
is subjected to interaction with thionyl chloride for the production of the corresponding chloranhydride of formula (VII) 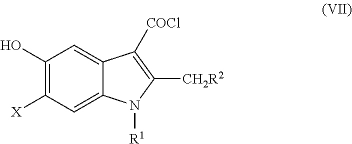
where R¹ and R² have the values indicated above,
which, without or after separation from the reaction mass, is subjected to interaction with a secondary amine, including the fragment N(R)₂.

6. A method according to claim 5, distinguished by the fact that a compound is used of formula VI,
obtained by saponification of a compound of formula (V) 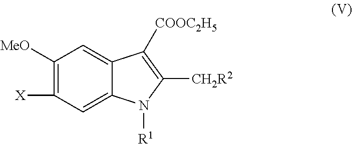
where X, R¹, and R² have the values indicated above.

7. A compound of general formula I for the production of a medicinal agent for the treatment of influenza A 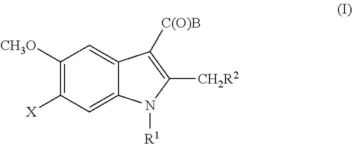
where X, R¹ R², and B are indicated above.
